(19) United States
(12) Patent Application Publication
(10) Pub. No.: US 2008/0029383 A1
(43) Pub. Date: Feb. 7, 2008

(54) Apparatus for detoxifying compositions containing heavy metal and a method of detoxification

(76) Inventor: Yasuhiro Sakakibara, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 11/641,732

(22) Filed: Dec. 20, 2006

(30) Foreign Application Priority Data
Jul. 25, 2006 (JP) JP2006-202363

(51) Int. Cl. C07C 1/00; B01J 19/08

(52) U.S. Cl. 204/157.15; 422/186

(57) ABSTRACT

An apparatus for detoxifying a composition containing heavy metal having a simple and downsized structure with improved reaction efficiency and reduced energy cost and a method of detoxification. The apparatus is provided with a reaction pipe, a storage container mounted to the reaction pipe for storing a composition containing heavy metal, a negative pressure producing means for producing a negative pressure by jetting a solution for eluting heavy metal to form a high-pressure fluid into the reaction pipe, a heater for heating a mixture obtained by mixing in the reaction pipe the high-pressure fluid and the composition containing heavy metal sucked in from the storage container into the reaction pipe by the negative pressure, the heater being disposed in the reaction pipe on a downstream side of the negative pressure producing means, and an electromagnetic wave irradiator for irradiating the mixture after heating with electromagnetic waves, the irradiator being disposed in the reaction pipe on a downstream side of the heater.

### BACKGROUND OF THE INVENTION

1. Field of the Invention

The present invention relates to an apparatus for detoxifying a composition containing heavy metal and a method of detoxification.

2. Description of the Related Art

Conventionally, several apparatuses for detoxifying a composition containing heavy metal such as incinerated ash of municipal waste or the like have been known. For example, it has been known that incinerated ash of municipal waste or the like is detoxified by mixing with an alkaline solution, followed by irradiation with electromagnetic waves while maintaining the temperature of the mixture at a predetermined level. The resultant composition can be effectively utilized as an artificial zeolite as disclosed in U.S. Pat. No. 6,663,845, for example.

A conventional apparatus for detoxifying incinerated ash is explained below. FIG. 3 is a schematic view illustrating a structure of a conventional apparatus for detoxifying incinerated ash. As shown in FIG. 3, a conventional detoxification apparatus 100 has a heating means 101 for adding an alkaline solution to incinerated ash as a raw material and heating the mixture, a mixing means 102 for preparing a slurry-form mixture by kneading the mixture, a conveyor 103 for carrying the resultant mixture toward a container for irradiation, and an electromagnetic wave irradiating means 104.

In the detoxification apparatus 100 as shown in FIG. 3, the heating step by the heating means 101, the mixing step by the mixing means 102, and the irradiation step by the electromagnetic wave irradiating means 104 are separately provided. Therefore, the temperature of the mixture is lowered by heat loss during the conveying step by the conveyor 103, which often causes separation of the alkaline solution from the mixture or coagulation of the mixture. Furthermore, the process divided into several steps complicates the reaction process, sometimes leading to deterioration of the reaction efficiency. In addition, the separate steps require a large-scale and complex apparatus as well as a larger amount of energy necessary to operate the apparatus.

In view of the foregoing, an object of the present invention is to provide an apparatus for detoxifying a composition containing heavy metal with a simplified and downsized structure, capable of enhancing reaction efficiency while reducing an energy cost and a method of detoxification.

### SUMMARY OF THE INVENTION

In order to solve the above-described problems, an apparatus for detoxifying a composition containing heavy metal according to the present invention comprises a reaction pipe, a storage container mounted to the reaction pipe for storing a composition containing heavy metal, a negative pressure producing means for producing a negative pressure by jetting a solution for eluting heavy metal from the composition to form a high-pressure fluid into the reaction pipe, a heater for heating a mixture obtained by mixing in the reaction pipe the high-pressure fluid and the composition containing heavy metal sucked in from the storage container into the reaction pipe by the negative pressure, the heater being disposed in the reaction pipe on a downstream side of the negative pressure producing means, and an electromagnetic wave irradiator for irradiating the mixture after heating with electromagnetic waves, the irradiator being disposed in the reaction pipe on a downstream side of the heater.

The solution for eluting heavy metal, or an acid or alkaline solution which is jetted into the reaction pipe to form the high-pressure fluid and the composition containing heavy metal sucked in by the negative pressure producing means flow through the reaction pipe while being mixed and stirred in the reaction pipe. The mixture is then heated by the heater and irradiated with electromagnetic waves by the electromagnetic wave irradiator to be detoxified. Thus, in the present invention, the heater and the electromagnetic wave irradiator are sequentially provided in the reaction pipe on the downstream side of the negative pressure producing means in this order. Accordingly, the present invention enables mixing and stirring the composition containing heavy metal and the solution for eluting heavy metal from the composition, heating the mixture, and irradiating the mixture with electromagnetic waves as a series of operations in the same pipe, which prevents heat loss during the detoxifying process and improves reaction efficiency to effectively detoxify the composition containing heavy metal. Moreover, since each of the steps is continuously conducted in the same pipe, the apparatus can be simplified and downsized.

Preferably, the negative pressure producing means may comprise a spray nozzle for jetting the solution for eluting heavy metal from the composition to form a high-pressure fluid into the reaction pipe, the spray nozzle having a bore diameter smaller than a diameter of the reaction pipe, and a fluoride injector provided in the reaction pipe for injecting fluoride into the high-pressure fluid jetted from the spray nozzle.

By jetting the high-pressure fluid of the solution for eluting heavy metal into the reaction pipe from the spray nozzle having a diameter smaller than a diameter of the reaction pipe, a negative pressure zone is generated on the downstream side of the spray nozzle. With the generated negative pressure zone, a composition containing heavy metal is sucked in from the storage container into the reaction pipe. Then, fluoride is injected to the high-pressure fluid jetted from the spray nozzle so that generation of cavitation of the high-pressure fluid can be prevented and the high-pressure fluid smashes against the composition containing heavy metal sucked into the reaction pipe without energy loss. In this way, the composition containing heavy metal, while being crushed into fine particles, is mixed with the solution for eluting heavy metal from the composition and forcibly flows toward a downstream side of the reaction pipe. By crushing the composition into fine particles, the total surface area of the composition containing heavy metal is increased, thereby enhancing the efficiency not only in the reaction with the solution but also in the reaction during the subsequent heating and electromagnetic wave irradiating steps in the reaction pipe. In addition, in the present invention, fluoride which is employed as a gas injected to prevent the generation of cavitation of the high-pressure fluid can promote the decomposition reaction of the composition containing heavy metal which is brought into contact with the solution, further improving the reaction efficiency.

Preferably, an apparatus of the present invention may further comprise an eluting means for eluting an unreacted substance which adheres to the mixture irradiated with electromagnetic waves by the electromagnetic wave irradiator.

By this structure, an unreacted substance adhering to the surface of the composition detoxified by the electromagnetic wave irradiation can be cleaned, leading to a high-purity resultant composition which does not contain heavy metal.

Furthermore, an apparatus of the present invention may preferably comprise a solid-liquid separator for separating the mixture irradiated with electromagnetic waves by the electromagnetic wave irradiator into a solid and a liquid. In addition, a supplying means for collecting a liquid component separated by the solid-liquid separator and supplying the liquid component to the spray nozzle may be provided.

By the above structure, a solid component separated from the reacted composition can be utilized as an aggregate, a material for embankment, a backfill material or the like. The liquid part which is collected and supplied to the spray nozzle or the eluting means enables repeated uses of the solution for eluting heavy metal from the composition in a closed cycle to provide the apparatus with less discharge of the waste liquid.

Preferably, the heater may be a planar heater.

A planar heater employed as a heating means is disposed to wrap the reaction pipe so that the mixture flowing through the reaction pipe can be directly and uniformly heated to provide the heater with high energy efficiency.

### BRIEF DESCRIPTION OF THE DRAWINGS

In the accompanying drawings:

FIG. 1 illustrates a general structure of an apparatus for detoxifying a composition containing heavy metal according to an embodiment of the present invention;

FIG. 2 is a sectional view of a mixing pipe; and

FIG. 3 is a schematic view illustrating a conventional apparatus for detoxifying a composition containing heavy metal.

### DETAILED DESCRIPTION OF THE PREFERRED EMBODIMENT

With reference to the accompanied drawings, an embodiment of the present invention will be explained below.

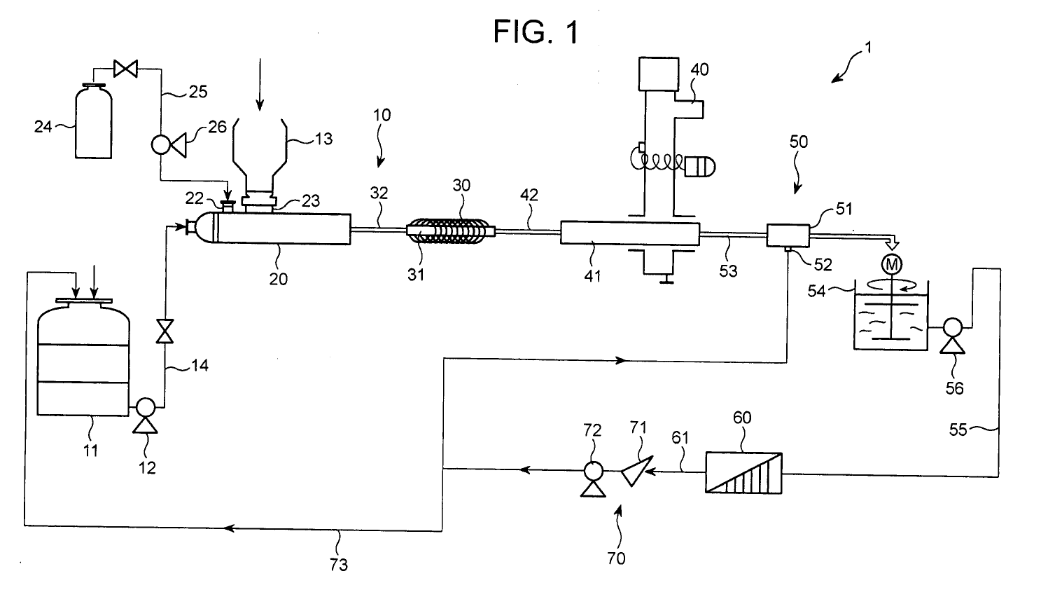

As shown in FIG. 1, a detoxification apparatus 1 for detoxifying a composition containing heavy metal in the present embodiment (referred to as “the detoxification apparatus” hereinafter) is provided with a storage tank 11 for storing a solution for eluting heavy metal from the composition (referred to as “the eluting solution” hereinafter), a storage container 13 for storing the composition containing heavy metal (referred to as “the composition” hereinafter), a mixing pipe 20 for mixing and stirring the eluting solution jetted by a high-pressure pump 12 (described later) and the composition sucked in from the storage container 13 by a negative pressure produced by the jetted eluting solution, a planar heater 30 for heating a mixture of the mixed and stirred composition and the eluting solution, an electromagnetic wave irradiator 40 for irradiating the heated mixture with electromagnetic waves, a cleaning device 50 as an eluting means for eluting an unreacted substance adhering to the irradiated mixture using the eluting solution, and a dehydrator 60 for separating the mixture irradiated with electromagnetic waves by the electromagnetic wave irradiator 40 and cleaned by the cleaning device 50 into a solid and a liquid.

The storage tank 11 is a known conventional tank to store an eluting solution provided with a coating on an inner peripheral surface of the tank to prevent corrosion by acid or alkali. The storage tank 11 and the mixing pipe 20 are connected to each other via a connecting tube 14, to which the high-pressure pump 12 is mounted. The high-pressure pump 12 may be any conventional high-pressure pump such as a plunger pump.

The storage container 13 stores a composition containing heavy metal, such as lime ash, incinerated ash of municipal waste, or asbestos, and any conventional container can be employed as the storage container 13. It is preferable that a composition to be stored in the storage container 13 is finely crushed using a crusher or the like in advance to remove foreign substances such as metal shards therefrom.

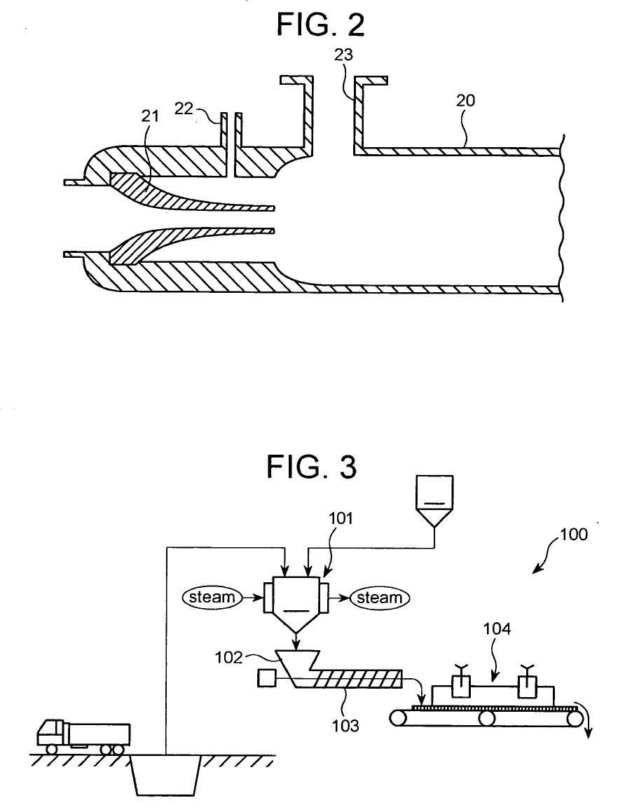

As shown in FIG. 2, the mixing pipe is provided with a spray nozzle 21 for jetting the eluting solution stored in the storage tank 11 into the mixing pipe 20, a fluoride gas feeding pipe 22 as an injecting means for injecting a fluoride gas into the high-pressure eluting solution jetted from the spray nozzle 21, and a suction port 23 for sucking in the composition stored in the storage container 13.

The spray nozzle 21 is formed to have a bore diameter which is smaller than a diameter of the mixing pipe 20. The eluting solution jetted in a form of high-pressure liquid by the high-pressure pump 12 into the mixing pipe 20 having a diameter which is larger than the diameter of the spray nozzle 21 generates a negative pressure zone on a downstream side of the spray nozzle 21 in the mixing pipe 20.

The fluoride gas feeding pipe 22 is provided on an upstream side of a distal end of the spray nozzle 21 in the mixing pipe 20 and connected to a fluoride gas tank 24 via a connection tube 25. The connection tube 25 has a pump 26 which adjusts a pressure of a fluoride gas stored in the fluoride gas tank 24 when injected into the mixing pipe 20. The fluoride gas stored in the fluoride gas tank 24, of which injecting pressure has been adjusted by the pump 26, is fed from the fluoride gas feeding pipe 22 into the mixing pipe 20 and flows around an outer periphery of the spray nozzle 21 toward a downstream side, preventing generation of cavitation of the eluting solution jetted from the spray nozzle 21 into the mixing pipe 20.

A planar heater 30 is mounted on a heating pipe 31 connected to the mixing pipe 20 via a tube 32 so as to wrap an outer periphery of the heating pipe 31. Any conventional planar heater can be employed as the planar heater 30 while a planar heater having a PTC (Positive Temperature Coefficient) characteristic is preferable. When a PTC planar heater is employed, the mixture flowing through the heating pipe 31 can be uniformly heated because a PTC planar heater has a uniform temperature distribution. The duration of heating the mixture with the planar heater 30 is adjusted by varying a flow velocity of the mixture with changing a pressure of the high-pressure pump 12 or by varying a heating area with changing the size and number of the planar heater 30 mounted on the heating pipe 31.

An electromagnetic wave irradiator 40 is mounted on an electromagnetic wave irradiating pipe 41 which is connected to the heating pipe 31 via a tube 42. The electromagnetic wave irradiator 40 irradiates the mixture flowing through the electromagnetic wave irradiating pipe 41 with electromagnetic waves having a wavelength approximately from 300 MHz to 30 GHz so that a hydrothermal reaction of the mixture is promoted to decompose and detoxify the composition containing heavy metal.

A cleaning device 50 has a cleaning pipe 51 connected to the electromagnetic wave irradiating pipe 41 via a tube 53, and a feeding port 52 provided on the cleaning pipe 51 for feeding the eluting solution. From the feeding port 52, an acid or alkaline eluting solution as cleaning water is jetted into the cleaning pipe 51. The cleaning device 50 jets the eluting solution to the mixture irradiated with electromagnetic waves by the electromagnetic wave irradiator 40, thereby eluting and removing an unreacted substance adhering to the mixture. The mixture from which the unreacted substance has been removed by the cleaning device 50 is temporarily stored in a slurry tank 54. The mixture stored in the slurry tank 54 is then carried to a dehydrator 60 by a pump 56 provided on a connecting tube 55.

The dehydrator 60 separates the mixture after removing the unreacted substance therefrom by the cleaning device 50 into a solid and a liquid. Any conventional device such as a centrifugal separator or a suction extractor can be used as the dehydrator 60. The dehydrator 60 is connected to a circulation unit 70 via a connecting tube 61. The circulation unit 70 collects a liquid component separated by the dehydrator 60 and supplies the liquid component to the storage tank 11 and the feeding port 52. The circulation unit 70 has a filter 71 such as a wedge wire screen (not shown) which removes a small solid component contained in the liquid component. The circulation unit 70 is connected to the storage tank 11 and the feeding port 52 via a branched tube 73 on each side, and the liquid component is supplied to the storage tank 11 and the feeding port 52, respectively, with a pump 72.

In the present embodiment, the mixing pipe 20, the heating pipe 31, the electromagnetic wave irradiating pipe 41, the cleaning pipe 51, and the tubes 32, 42, and 53 function as the reaction pipe 10 as a whole.

Next, a process for detoxifying a composition in the present embodiment will be explained below in detail.

The eluting solution is jetted in a form of high-pressure liquid from the spray nozzle 21 into the mixing pipe 20 to generate a negative pressure zone on a downstream side of the spray nozzle 21. The negative pressure zone thus generated causes a composition stored in the storage container 13 to be sucked in from the suction port 23 into the mixing pipe 20.

Here, a fluoride gas, of which pressure has been adjusted, is injected from the fluoride gas feeding pipe 22 to the eluting solution jetted from the spray nozzle 21 into the mixing pipe 20. Thus, generation of cavitation of the eluting solution jetted from the spray nozzle 21 is prevented, and the eluting solution smashes against the sucked composition without energy loss. In this way, the composition is mixed with the eluting solution and forcibly flows toward a downstream side while being subjected to shock crushing into fine particles. The total surface area of the composition increases by fine crushing, thereby enhancing reaction efficiency with the eluting solution in the mixing pipe 20. In addition, the fluoride gas injected into the mixing pipe 20 further crushes the composition into fine particles and stirs the composition, which increasingly promotes a hydrothermal reaction of the composition.

The mixture which is mixed with the eluting solution and the fluoride gas and forcibly flows within the mixing pipe 20 further goes through the mixing pipe 20 while being further crushed into fine particles by interparticle collision caused among the mixtures. Then, the mixture flows via the tube 32 into the heating pipe 31 provided with the planar heater 30.

The planar heater 30 heats the mixture to a temperature range at which a hydrothermal reaction is enhanced or, in particular, approximately from 30 to 180 degrees centigrade. The mixture, while maintaining the temperature, flows through the tube 42 into the electromagnetic wave irradiating pipe 41 provided with the electromagnetic wave irradiator 40. The mixture which has reached the electromagnetic wave irradiating pipe 41 is irradiated with electromagnetic waves by the electromagnetic wave irradiator 40 to promote a hydrothermal reaction due to heat generated from the inside of the particles of the mixture, thereby detoxifying the mixture in a short time. Since the mixture has already been crushed into fine particles during the process of flowing through the mixing pipe 20 on the upstream side, the increased total surface area of the composition leads to high reaction efficiency during the heating step by the planar heater 30 and the electromagnetic irradiation step by the electromagnetic wave irradiator 40 as well.

The mixture irradiated with electromagnetic waves by the electromagnetic wave irradiator 40 further flows toward a downstream side. Then, an unreacted substance adheres to a surface of the mixture. However, as the mixture flows to the downstream side while being further mixed with the fluoride gas fed into the mixing pipe 20 by the fluoride gas feeding pipe 22, the unreacted substance is thereby eluted to clean the mixture. In the cleaning device 50 provided on a downstream side of the electromagnetic wave irradiator 40, the mixture is again cleaned by the eluting solution jetted from the feeding port 52. Accordingly, the unreacted substance adhering to the surface of the mixture is almost perfectly removed to provide a highly pure reacted substance with no heavy metal contained.

As described above, according to the present embodiment, following the reaction pipe 10 as a continuous pipe comprising the mixing pipe 20, the heating pipe 31, the electromagnetic wave irradiating pipe 41, the cleaning pipe 51 and the tubes 32, 42, 53, provided are, from the upstream side to the downstream side, the spray nozzle 21 for producing a negative pressure, the planar heater 30 for heating the mixture, and the electromagnetic wave irradiator 40 for irradiating the mixture with electromagnetic waves in this order. By this structure, mixing and stirring the composition with the eluting solution, heating the mixture, and irradiating the mixture with electromagnetic waves can be conducted as a series of operations in the same pipe. Consequently, the detoxification apparatus 1 can be simplified and downsized while improving reaction efficiency, resulting in a reduced energy cost.

In order to enhance production of a negative pressure on a downstream side of the spray nozzle 21 as well as to facilitate a conveying operation of the mixture mixed in the mixing pipe 20, the mixing pipe 20 can be slantingly positioned so as to put the downstream side higher than the upstream side, or a connecting tube having a reverse U-shape can be connected to the mixing pipe 20 on the downstream side. By this structure, as mixing of the eluting solution and the composition progresses in the mixing pipe 20, the mixture gradually becomes apt to clog the mixing pipe 20 on the downstream side, which facilitates production of the negative pressure on the downstream side of the spray nozzle 21. At the same time, the jetting pressure of the eluting solution jetted from the spray nozzle 21 can forcibly carry the mixture away to the downstream side of the mixing pipe 20 with a stronger pressure.

The mixture from which the unreacted substance has been almost completely removed is separated into a solid and a liquid by the dehydrator 60. By the solid-liquid separation with the dehydrator 60, a separated solid substance can be utilized as an aggregate, a material for embankment, a backfill material, or the like. A liquid component is collected by the circulation unit 70 to remove a fine solid component therefrom with the filter 71 and then supplied into the storage tank 11 and the feeding port 52 via the branched tube 73. Thus, the eluting solution used in the detoxification apparatus 1 of the present embodiment can be repeatedly used by circulating in the closed cycle to reduce discharge of a waste liquid. In order to remove impurities adhering to the mixture, a step for cleaning the mixture with water may be added before the solid-liquid separation by the dehydrator 60.

Here, so as to ensure a sufficient reaction time of the mixture irradiated with electromagnetic waves by the electromagnetic wave irradiator 40, a curing device having a stirrer for stirring the mixture or an electromagnetic wave irradiator may be provided between the electromagnetic wave irradiator 40 and the cleaning device 50 or on the downstream side of the cleaning device 50.

Examples of the composition to be detoxified by the detoxification apparatus 1 of the present embodiment are lime ash, incinerated ash, asbestos, or the like. When using lime ash or incinerated ash as the composition, an alkaline solution can be employed as the eluting solution to obtain an artificial zeolite as a reacted substance after detoxification. In this case, the resultant artificial zeolite may be further processed to have a variety of functions in a post-process to obtain a functional artificial zeolite. When asbestos is employed as the composition, an acid is used as the eluting solution.

While there has been described what is at present considered to be a preferred embodiment of the invention, it will be understood that various modifications may be made thereto, and it is intended that the appended claims cover all such modifications as fall within the true spirit and scope of the invention.

1. An apparatus for detoxifying a composition containing heavy metal comprising:
a reaction pipe;
a storage container mounted to the reaction pipe for storing a composition containing heavy metal;
a negative pressure producing means for producing a negative pressure by jetting a solution for eluting heavy metal from the composition to form a high-pressure fluid into the reaction pipe;
a heater for heating a mixture obtained by mixing in the reaction pipe the high-pressure fluid and the composition containing heavy metal sucked in from the storage container into the reaction pipe by the negative pressure, the heater being disposed in the reaction pipe on a downstream side of the negative pressure producing means; and
an electromagnetic wave irradiator for irradiating the mixture after heating with electromagnetic waves, the irradiator being disposed in the reaction pipe on a downstream side of the heater.

2. The apparatus according to claim 1, wherein said negative pressure producing means comprises:
a spray nozzle for jetting the solution for eluting heavy metal from the composition to form a high-pressure fluid into the reaction pipe, the spray nozzle having a bore diameter smaller than a diameter of the reaction pipe; and
a fluoride injector provided in the reaction pipe for injecting fluoride into the high-pressure fluid jetted from the spray nozzle.

3. The apparatus according to claim 1, further comprises:
eluting means for eluting an unreacted substance which adheres to the mixture irradiated with electromagnetic waves by the electromagnetic wave irradiator.

4. The apparatus according to claim 2, further comprises:
eluting means for eluting an unreacted substance which adheres to the mixture irradiated with electromagnetic waves by the electromagnetic wave irradiator.

5. The apparatus according to claim 1, further comprises:
a solid-liquid separator for separating the mixture irradiated with electromagnetic waves by the electromagnetic wave irradiator into a solid and a liquid.

6. The apparatus according to claim 2, further comprises:
a solid-liquid separator for separating the mixture irradiated with electromagnetic waves by the electromagnetic wave irradiator into a solid and a liquid.

7. The apparatus according to claim 3, further comprises:
a solid-liquid separator for separating the mixture irradiated with electromagnetic waves by the electromagnetic wave irradiator into a solid and a liquid.

8. The apparatus according to claim 4, further comprises:
a solid-liquid separator for separating the mixture irradiated with electromagnetic waves by the electromagnetic wave irradiator into a solid and a liquid.

9. The apparatus according to claim 5, further comprises:
supplying means for collecting a liquid component separated by the solid-liquid separator and supplying the liquid component to the spray nozzle or the eluting means.

10. The apparatus according to claim 1, wherein said heater is a planar heater.

11. A method of detoxifying a composition containing heavy metal comprising:
storing a composition containing heavy metal in a storage container which is connected to a reaction pipe;
jetting a solution for eluting heavy metal from the composition to form a high-pressure fluid into the reaction pipe to produce a negative pressure;
mixing the composition sucked in from the storage container into the reaction pipe with the solution in the reaction pipe by the negative pressure to obtain a mixture;
heating the mixture; and
irradiating the heated mixture with electromagnetic waves.

12. The method according to claim 11, further comprising:
eluting an unreacted substance which adheres to the mixture irradiated with electromagnetic waves with a solution for eluting heavy metal.

13. The method according to claim 11, further comprising:
separating the mixture irradiated with electromagnetic waves into a solid and a liquid.

14. The apparatus according to claim 6, further comprises:
supplying means for collecting a liquid component separated by the solid-liquid separator and supplying the liquid component to the spray nozzle or the eluting means.

15. The apparatus according to claim 7, further comprises:
supplying means for collecting a liquid component separated by the solid-liquid separator and supplying the liquid component to the spray nozzle or the eluting means.

16. The apparatus according to claim 8, further comprises:
supplying means for collecting a liquid component separated by the solid-liquid separator and supplying the liquid component to the spray nozzle or the eluting means.
